(19) United States
(12) Patent Application Publication
(10) Pub. No.: US 2022/0313252 A1
(43) Pub. Date: Oct. 6, 2022

(54) SURGICAL STAPLING DEVICE WITH CARTRIDGE ASSEMBLY RETAINER

(71) Applicant: Covidien LP, Mansfield, MA (US)

(72) Inventor: Syed Sarfraz Ahamed, Shanghai (CN)

(21) Appl. No.: 17/639,410

(22) PCT Filed: Sep. 16, 2019
(86) PCT No.: PCT/CN2019/105928

§ 371 Date: Mar. 1, 2022

(51) Int. Cl. A61B 17/072

(52) U.S. Cl. CPC A61B 17/072; A61B 2017/07257; A61B 2017/07271

(57) ABSTRACT

A surgical stapling device (10) includes a clamp slide assembly (30) and a tool assembly (16). The tool assembly (16) includes an anvil assembly (22) and a cartridge assembly (20). The cartridge assembly (20) is releasably supported on the clamp slide assembly (30) and the clamp slide assembly (30) is movable in relation to the anvil assembly (22) to move the surgical stapling device (10) between spaced and clamped positions. The cartridge assembly (20) and the clamp slide assembly (30) each include retention structure that are movable into engagement with each other when the cartridge assembly (20) is loaded onto the clamp slide assembly (30) to retain the cartridge assembly (20) on the clamp slide assembly (30).

### FIELD

The present technology is generally related to surgical stapling devices and, more particularly, to surgical stapling devices with loadable cartridges.

### BACKGROUND

Surgical stapling devices for applying rows of staples through compressed living tissue are known in the art and are commonly used for closure of tissue or organs prior to transection or resection and for occlusion of organs during thoracic or abdominal procedures. Such stapling devices include a knife for transecting or resecting the tissue or organs simultaneous with the application of the rows of staples to the tissue.

Typically, stapling devices include a tool assembly including an anvil assembly and a cartridge assembly, a drive member or pusher, and an approximation assembly for moving the tool assembly between spaced and clamped positions. The tool assembly is available in a variety of configurations including linear, curved, and circular. In addition, the tool assembly is available in a variety of orientations in relation to a longitudinal axis of the stapling device including parallel and transverse orientations.

In transverse type stapling devices, the approximation mechanism of the stapling device typically includes a clamp slide mechanism that has a distal portion that supports a loadable cartridge assembly. The distal portion of the clamp slide mechanism is configured to allow a cartridge assembly to be slid onto the distal portion from one side of the clamp slide mechanism such that the cartridge assembly is movable with the clamp slide assembly from a retracted position spaced from the anvil assembly to the clamped position in juxtaposed alignment with the anvil assembly.

A continuing need exists for a stapling device including a simple retention mechanism capable of securely retaining the cartridge assembly on the cartridge receiving portion of the clamp slide mechanism.

### SUMMARY

The aspects of this disclosure generally relate to a surgical stapling device including a removable or releasable cartridge assembly. The surgical stapling device and the cartridge assembly include retention structure to retain the cartridge assembly on the surgical stapling device.

One aspect of the disclosure is directed to a stapling device including a frame assembly, a clamp slide assembly, and a tool assembly. The frame assembly defines a longitudinal axis and a frame assembly channel and includes a proximal portion and a distal portion. The clamp slide assembly is supported within the frame assembly channel and has a distal end portion and at least one post extending from the distal end portion of the clamp slide assembly. The tool assembly includes an anvil assembly and a cartridge assembly. The anvil assembly is supported on the distal portion of the frame assembly. The cartridge assembly is releasably supported on the distal end portion of the clamp slide assembly and includes a cartridge body having spaced side walls. The spaced side walls define a cartridge channel that is dimensioned to engage the distal end portion of the clamp slide assembly. Each of the spaced side walls define at least one slot that receives the at least one post of the clamp slide assembly when the cartridge assembly is received on the clamp slide assembly to retain the cartridge assembly on the clamp slide assembly.

In aspects of the disclosure, the at least one post includes two posts on each side of the clamp slide assembly and the at least one slot includes two slots formed in each of the spaced side walls of the cartridge body.

In some aspects of the disclosure, each of the at least one slot is defined by angled walls that converge in a distal direction into a circular hole.

In certain aspects of the disclosure, each of the circular holes of each of the slots includes an entryway having a width, and each of the at least one post has a diameter that is larger than the width of the entryway such that receipt of the at least one post within the circular hole of the at least one slot causes deformation of the side walls of the cartridge body.

In aspects of the disclosure, the proximal portion of the frame assembly supports a handle assembly.

In some aspects of the disclosure, the handle assembly includes a stationary handle and a trigger that is movable in relation to the stationary handle to actuate the tool assembly.

In certain aspects of the disclosure, the clamp slide assembly includes spaced clamp members having distal end portions and the distal end portions of the spaced clamp members are secured together with rivets, wherein the rivets include a central portion having a first diameter and opposite end portions having a second diameter that is smaller than the first diameter.

In aspects of the disclosure, the opposite end portions of the rivets form the posts.

Another aspect of the disclosure is directed to a stapling device including a frame assembly, a handle assembly, a clamp slide assembly, and a tool assembly. The frame assembly defines a longitudinal axis and includes spaced frame members that define a frame assembly channel. Each of the spaced frame members has a proximal portion, a central portion, and a distal portion. The handle assembly is supported on the proximal portion of the frame members. The central portion of the frame members defines an elongate body that extends distally from the handle assembly. The clamp slide assembly is supported within the frame assembly channel and extends from the handle assembly along the elongate body. The clamp slide assembly includes spaced clamp members. Each of the spaced clamp members has a distal end portion and at least one post extending outwardly from the respective clamp member. The tool assembly includes an anvil assembly and a cartridge assembly. The anvil assembly is supported on the distal portions of the frame members. The cartridge assembly is releasably supported on the distal end portions of the clamp members and includes a cartridge body having spaced side walls. The spaced side walls define a cartridge channel that is dimensioned to receive the distal end portions of the spaced clamp members. Each of the spaced side walls also defines at least one slot that receives the at least one post of the clamp slide assembly when the cartridge assembly is received on the clamp slide assembly to retain the cartridge assembly on the clamp slide assembly.

Another aspect of the disclosure is directed to a stapling device including a frame assembly, a clamp slide assembly, and a tool assembly. The frame assembly defines a longitudinal axis and includes spaced frame members that define a frame assembly channel. Each of the spaced frame members has a proximal portion and a distal portion. The clamp slide assembly is supported within the frame assembly channel and includes spaced clamp members. Each of the spaced clamp members has a distal end portion and at least one post extending outwardly from the respective clamp member. The tool assembly includes an anvil assembly and a cartridge assembly. The anvil assembly is supported on the distal portions of the frame members. The cartridge assembly is releasably supported on the distal end portion of the clamp members and includes a cartridge body having spaced side walls. The spaced side walls define a cartridge channel that is dimensioned to receive the distal end portions of the spaced clamp members. Each of the spaced side walls define at least one slot that receives the at least one post of the clamp slide assembly when the cartridge assembly is received on the clamp slide assembly to retain the cartridge assembly on the clamp slide assembly. The at least one slot is defined by angled walls that converge in a distal direction into a circular hole. Each of the circular holes of the at least one slot includes an entryway having a width, and each of the at least one post has a diameter that is larger than the width of the entryway such that receipt of the at least one post within the circular hole of the at least one slot causes deformation of the side walls of the cartridge body.

Other features of the disclosure will be appreciated from the following description.

### BRIEF DESCRIPTION OF DRAWINGS

Various aspects of the disclosure are described herein below with reference to the drawings, wherein:

FIG. 1 is a side perspective view of a surgical stapling device with a tool assembly in a spaced position illustrating certain aspects of the disclosure including a locking device;

FIG. 2 is a side perspective view a clamp slide assembly of the stapling device shown in FIG. 1 with clamp slide members of the clamp slide assembly separated from each other;

FIG. 3 is a side perspective view of the clamp slide assembly shown in FIG. 2 assembled and frame members of the stapling device shown in FIG. 1 separated from each other;

FIG. 4 is a side perspective view of a distal portion of the stapling device shown in FIG. 1 including the cartridge assembly and the anvil assembly prior to insertion of the cartridge assembly into the clamp slide assembly;

FIG. 5 is a side perspective view of the distal portion of the stapling device shown in FIG. 4 with the cartridge assembly received on the clamp slide assembly in an unlocked position;

FIG. 6 is an enlarged view of the indicated area of detail shown in FIG. 5;

FIG. 7 is a side perspective view of the distal portion of the stapling device shown in FIG. 5 with the cartridge assembly received on the clamp slide assembly as the cartridge assembly is moved towards the locked position;

FIG. 8 is an enlarged view of the indicated area of detail shown in FIG. 7;

FIG. 9 is a side perspective view of the distal portion of the stapling device shown in FIG. 5 with the cartridge assembly received on the clamp slide assembly in the locked position;

FIG. 10 is an enlarged view of the indicated area of detail shown in FIG. 9;

FIG. 11 is an enlarged view of the indicated area of detail shown in FIG. 1; and

FIG. 12 is a side perspective view of the distal portion of the stapling device shown in FIG. 1 with the cartridge assembly in the locked position.

### DETAILED DESCRIPTION

The disclosed device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. In addition, directional terms such as front, rear, upper, lower, top, bottom, distal, proximal, and similar terms are used to assist in understanding the description and are not intended to limit the present disclosure.

In this description, the term “proximal” is used generally to refer to that portion of the device that is closer to a clinician, while the term “distal” is used generally to refer to that portion of the device that is farther from the clinician. In addition, the term “endoscopic” is used generally used to refer to endoscopic, laparoscopic, arthroscopic, and/or any other procedure conducted through small diameter incision or cannula. Further, the term “clinician” is used generally to refer to medical personnel including doctors, nurses, and support personnel.

The disclosed surgical stapling device has a clamp slide assembly and a tool assembly that includes an anvil assembly and a cartridge assembly. The cartridge assembly is releasably supported on the clamp slide assembly and the clamp slide assembly is movable in relation to the anvil assembly to move the surgical stapling device between spaced and clamped positions. The cartridge assembly and the clamp slide assembly each include retention structure that are movable into engagement with each other when the cartridge assembly is loaded onto the clamp slide assembly to retain the cartridge assembly on the clamp slide assembly.

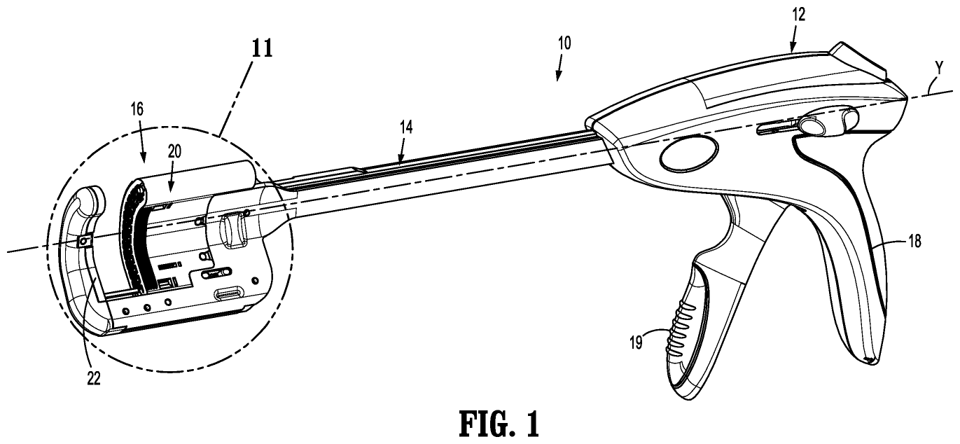

FIG. 1 illustrates the disclosed surgical stapling device shown generally as stapling device 10. The stapling device 10 includes a handle assembly 12, an elongate body 14 that defines a longitudinal axis “Y”, and a tool assembly 16. The handle assembly 12 includes a stationary handle 18 and a trigger 19 that is movable in relation to the stationary handle 18 to actuate the tool assembly 16. The tool assembly 16 includes a staple cartridge assembly 20 and an anvil assembly 22 that define axes that are transverse to the longitudinal axis “Y” of the elongate body 14. The cartridge assembly 20 is supported on a distal end portion of the elongate body 14 and is movable in relation to the anvil assembly 22 between a spaced position and a clamped position. In the clamped position, the cartridge assembly 20 is in juxtaposed alignment with the anvil assembly 22. In some aspects of the disclosure, the cartridge assembly 20 and the anvil assembly 22 are curved to facilitate access to certain anatomical regions of a patient.

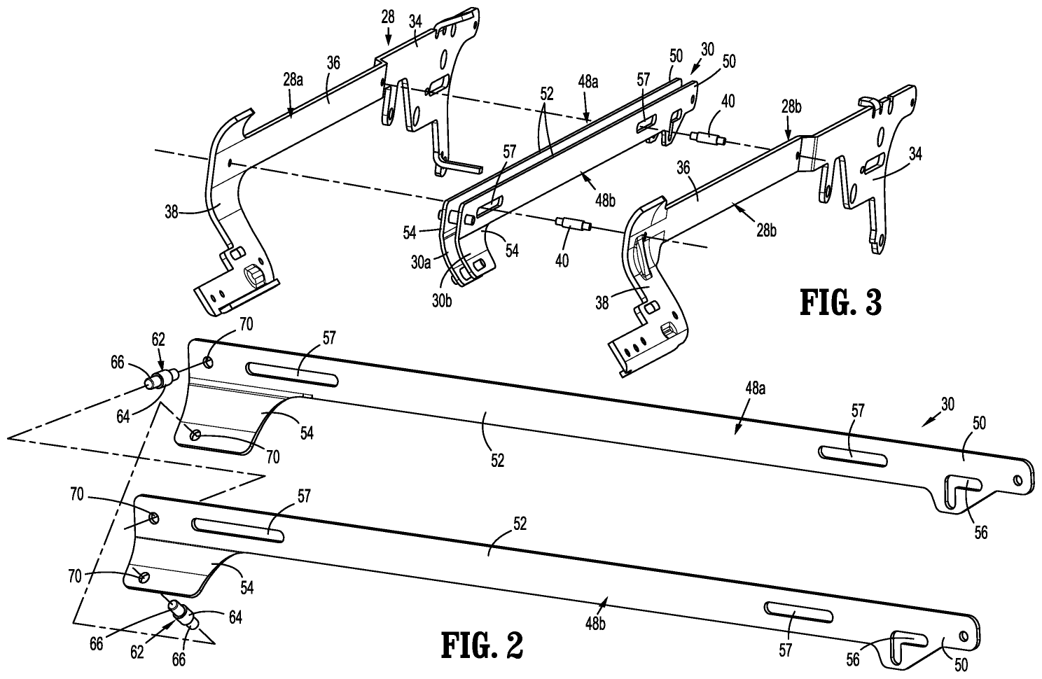

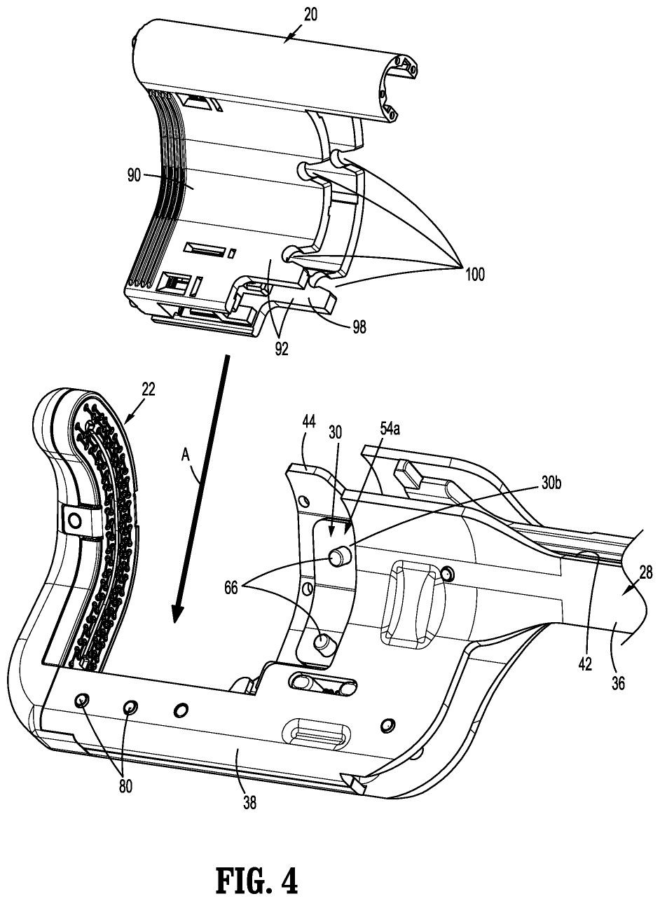

FIGS. 2 and 3 illustrate a frame assembly 28 and a clamp slide assembly 30 of the stapling device 10. The frame assembly 28 defines a longitudinal axis and includes first and second spaced frame members 28a and 28b. Each of the frame members 28a and 28b includes a proximal portion 34 that forms a portion of the handle assembly 12, a central portion 36 that forms a portion of the elongate body 14, and a distal portion 38 that forms a portion of the tool assembly 16. The frame members 28a and 28b are secured together with rivets including rivets 40 (FIG. 3) in spaced relation to each other to define a frame assembly channel 42 (FIG. 4) between the frame members 28a and 28b. The channel 42 receives the clamp slide assembly 30 and a thrust bar 44 (FIG. 4). The thrust bar 44 is provided to facilitate firing of staples (not shown) from the cartridge assembly 20 into the anvil assembly 22. For a more detailed description of the handle assembly 12 and operation of the thrust bar 44, see U.S. Pat. No. 6,817,508 (“the '508 Patent”). It is envisioned that the frame assembly could be formed of a single component rather two components as described above.

The clamp slide assembly 30 is received within the channel 42 (FIG. 4) of the frame assembly 28 and includes spaced clamp members 48a and 48b that are secured together in spaced relation to each other. Each of the clamp members 48a and 48b includes a proximal end portion 50, a central portion 52, and a distal end portion 54. The proximal end portion 50 of each of the clamp members 48a and 48b defines a cam slot 56 that forms a component of an alignment pin advancement assembly (not shown). For a more detailed description of the alignment pin advancement assembly, see the '508 Patent. The central portion 52 and the distal end portion 54 of each clamp members 48a and 48b defines an elongate slot 57. The elongate slots 57 receive the rivets 40 (FIG. 3) of the frame assembly 28 to limit the clamp slide assembly 30 to linear movement within the channel 42 of the frame assembly 28. The distal end portion 54 of each of the clamp members 48a and 48b has an enlarged width. When the clamp members 48a and 48b are secured together, the distal end portions 54 of the clamp members 48a and 48b provide a support structure for the cartridge assembly 20 as described in further detail below. It is envisioned that the clamp slide assembly 30 could be formed of a single component instead of two separate components as described above.

The distal end portions 54 of the clamp members 48a and 48b are connected together by rivets 62 (FIG. 2). The rivets 62 have central portion 64 that has a first diameter and opposite end portions 66 that have a second diameter that is less than the first diameter. The opposite end portions 66 of the rivets 62 are received within openings 70 formed in the distal end portions 54 of the clamp members 48a and 48b. The openings 70 have a diameter to receive the opposite ends 66 of the rivets 62 but to prevent passage of the central portion 64 of the rivets 62. As such, the length of the central portion 64 of the rivets 62 defines the spacing between the clamp members 48a and 48b. The opposite ends 66 of the rivets 62 have a length greater than the thickness of the clamp members 48a and 48b and extend outwardly from the distal end portions 54 of the clamp members 48a and 48b to define posts 66 (FIG. 4). The posts 66 extend in a direction transverse to the longitudinal axis of the frame assembly 28. It is envisioned that the posts 66 of the clamp slide assembly 30 can be formed separately from the rivets 62 and may extend inwardly or outwardly from the clamp members 48a and 48b depending on how the cartridge assembly 20 is received on the clamp slide assembly 30.

The anvil assembly 22 is secured to the distal portion 38 of the frame members 28a and 28b. In aspects of the disclosure, rivets, screws, or the like 80 (FIG. 4) are used to secure the anvil assembly 22 to the frame members 28a and 28b. Alternately, other fastening techniques or devices can be used to secure the anvil assembly 22 to the frame assembly 28.

FIG. 4 illustrates the cartridge assembly 20 separated from the clamp slide assembly 30. The cartridge assembly 20 includes a body 90 having spaced side walls 92, at least one pusher (not shown), a knife and knife support (not shown), and a plurality of staples (not shown). The side walls 92 define a channel 98 that is open at one end and is dimensioned to receive or engage the distal end portions 54 of the clamp slide assembly 30. Each of the side walls 92 includes a proximal portion that defines two slots 100 that receive the opposite end portions or posts 66 of the rivets 62 when the cartridge assembly 30 is supported on the clamp slide assembly 30.

FIG. 6 illustrates an enlarged view of a slot 100 which is defined by angled walls 102 that converge in a distal direction into a circular hole 104 that is dimensioned to receive one of the posts 66. In some aspects of the disclosure, the angled walls 102 define an entryway to the circular hole 104 that has a dimension “X” (FIG. 6) that is slightly smaller than the diameter of the respective post 66. When the post 66 engages the angled walls 102, the side walls 92 are deformed slightly outwardly of the post 66 to allow entry of the posts 66 into the circular hole 104. After the post 66 enters the circular hole 104, the side walls 92 of the cartridge body 90 return to their original undeformed positions to securely retain the cartridge assembly 22 on the distal end of the clamp slide assembly 30.

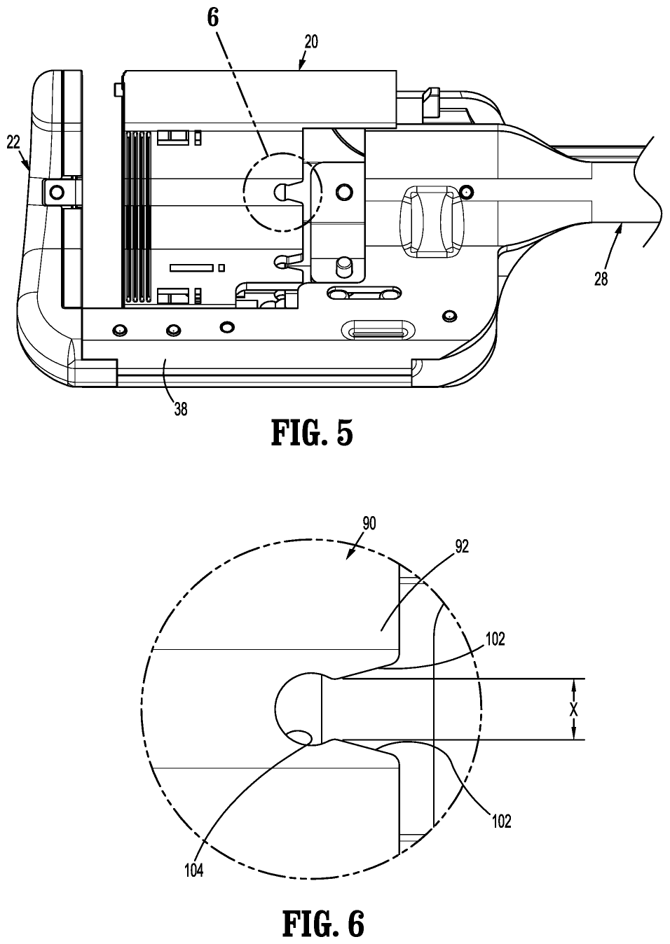

FIGS. 4-6 illustrate the tool assembly 16 as the cartridge assembly 20 is slid in the direction indicated by the arrow “A” in FIG. 4 (transverse to the longitudinal axis “Y” of the elongate body 14) onto the clamp slide assembly 30. When the cartridge assembly 20 is slid onto the clamp slide assembly 30, the channel 98 is positioned to face the distal portion 38 of the frame assembly 28 and the side walls 92 of the cartridge body 90 are positioned about the distal end portion 54 of the clamp members 30a and 30b (FIG. 3). The cartridge assembly 20 is slid in the direction of arrow “A” towards the distal portion 38 of the frame assembly 28 until the opposite end portions 66 of the rivets 62 are aligned with the slots 100. It is envisioned that the distal end portion 54a of the clamp slide assembly 30 can be configured to receive the cartridge body 90 of the cartridge assembly 20 in contrast to being received within the cartridge body 90 as illustrated.

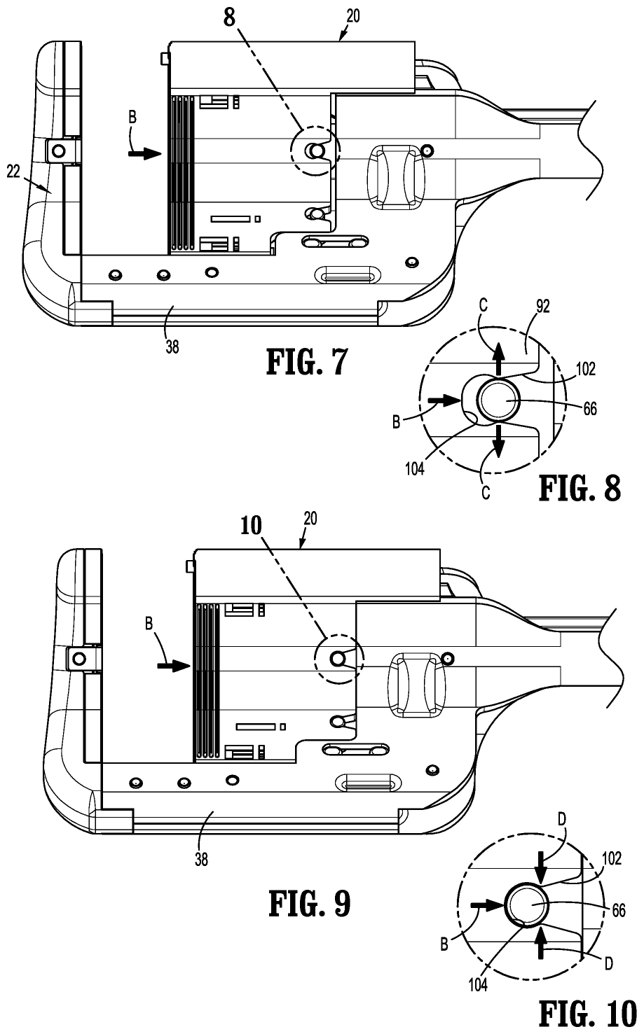

When the opposite end portions 66 of the rivets 62 are aligned with the slots 100, the cartridge assembly 20 is slid longitudinally in the direction of arrow “B” in FIGS. 7 and 9 to position the posts 66 within the holes 104 of the slots 100. As described above and shown in FIG. 8, the outer diameters of the posts are slightly larger than the dimension “X” of the entryway into the circular hole 104 of the slot 100. As such, the side walls 92 of the cartridge body 90 flex outwardly in the direction of arrows “C” as the posts enter the hole 104. Similarly, the side walls 92 return to their original configuration in the direction of arrows “D” upon receipt of the posts 66 into the holes 104 (FIG. 10). This securely retains the cartridge assembly 20 onto the clamp slide assembly 30. The angled walls 102 defining the entryway into the slots 100 properly align the cartridge assembly 20 with the clamp slide assembly 30.

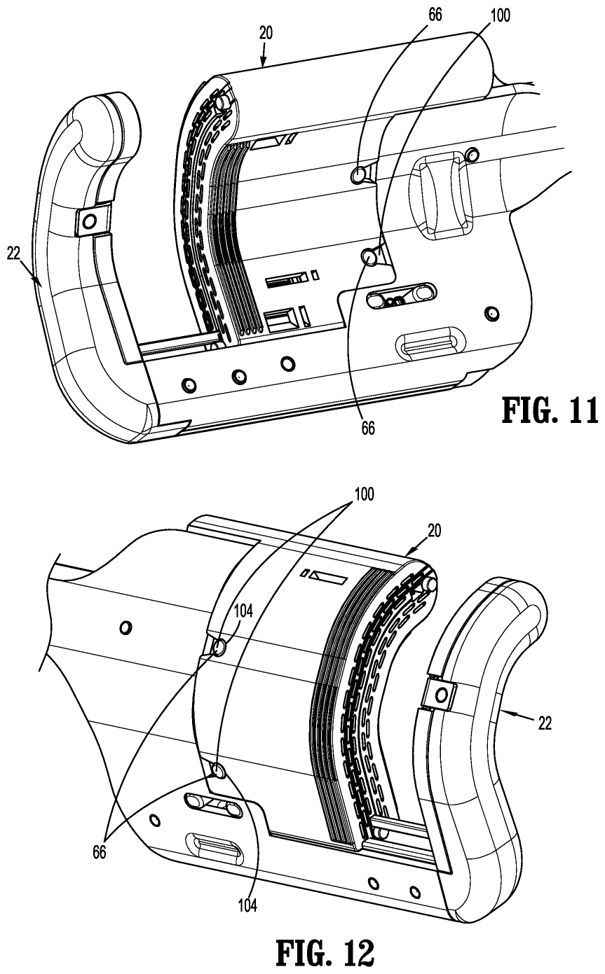

FIGS. 11 and 12 illustrate the cartridge assembly 20 supported on the clamp slide assembly 30 (FIG. 3) in a locked position. In the locked position, the posts 66 are received fully within the holes 104 of the slots 100 in the cartridge body 90 to securely retain the cartridge assembly 20 on the clamp slide assembly 30. After the stapling device 10 is fired, the cartridge assembly 30 can be removed from the clamp slide assembly 30 by applying a force to the cartridge assembly 20 in a direction opposite to the direction of arrow “B” in FIG. 9 that is sufficient to remove the posts 66 from the holes 104 and then sliding the cartridge assembly 20 in the direction opposite to the direction of arrow “A” in FIG. 4.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

1. A stapling device comprising:
a frame assembly defining a longitudinal axis and a frame assembly channel, the frame assembly having a proximal portion and a distal portion;
a clamp slide assembly supported within the frame assembly channel, the clamp slide assembly having a distal end portion and at least one post extending from the distal end portion of the clamp slide assembly; and
a tool assembly including an anvil assembly and a cartridge assembly, the anvil assembly supported on the distal portion of the frame assembly, the cartridge assembly being releasably supported on the distal end portion of the clamp slide assembly and including a cartridge body having spaced side walls, the spaced side walls defining a cartridge channel dimensioned to engage the distal end portion of the clamp slide assembly, each of the spaced side walls defining at least one slot that receives the at least one post of the clamp slide assembly when the cartridge assembly is received on the clamp slide assembly to retain the cartridge assembly on the clamp slide assembly.

2. The stapling device of claim 1, wherein the at least one post includes two posts on each side of the clamp slide assembly and the at least one slot includes two slots formed in each of the spaced side walls of the cartridge body.

3. The stapling device of claim 1, wherein each of the at least one slot is defined by angled walls that converge in a distal direction into a circular hole.

4. The stapling device of claim 3, wherein each of the circular holes of each of the slots includes an entryway having a width, each of the at least one post has a diameter that is larger than the width of the entryway such that receipt of the at least one post within the circular hole of the at least one slot causes deformation of the side walls of the cartridge body.

5. The stapling device of claim 1, wherein the proximal portion of the frame assembly supports a handle assembly.

6. The stapling device of claim 1, wherein the handle assembly includes a stationary handle and a trigger, the trigger movable in relation to the stationary handle to actuate the tool assembly.

7. The stapling device of claim 1, wherein the clamp slide assembly includes a pair of spaced clamp members having distal end portions, the distal end portions of the spaced clamp members being secured together with rivets, each of the rivets including a central portion having a first diameter and opposite end portions having a second diameter smaller than the first diameter.

8. The stapling device of claim 7, wherein the opposite end portions of the rivets form the at least one post.

9. A stapling device comprising:
a frame assembly defining a longitudinal axis and including spaced frame members the spaced frame members that define a frame assembly channel, each of the spaced frame members having a proximal portion, a central portion, and a distal portion;
a handle assembly supported on the proximal portion of the frame members, the central portion of the frame members defining an elongate body that extends distally from the handle assembly;
a clamp slide assembly supported within the frame assembly channel and extending from the handle assembly along the elongate body, the clamp slide assembly including spaced clamp members, each of the spaced clamp members having a distal end portion and at least one post extending outwardly from the respective clamp member; and
a tool assembly including an anvil assembly and a cartridge assembly, the anvil assembly supported on the distal portions of the frame members, the cartridge assembly releasably supported on the distal end portions of the clamp members and including a cartridge body having spaced side walls, the spaced side walls defining a cartridge channel dimensioned to receive the distal end portions of the spaced clamp members, each of the spaced side walls defining at least one slot that receives the at least one post of the clamp slide assembly when the cartridge assembly is received on the clamp slide assembly to retain the cartridge assembly on the clamp slide assembly.

10. The stapling device of claim 9, wherein the at least one post includes two posts on each of the spaced clamp members and the at least one slot includes two slots formed in each of the spaced side walls of the cartridge body.

11. The stapling device of claim 10, wherein each of the at least one slots is defined by angled walls that converge in a distal direction into a circular hole.

12. The stapling device of claim 11, wherein each of the circular holes of each of the at least one slot includes an entryway having a width, each of at least one post has a diameter that is larger than the width of the entryway such that receipt of the at least one post within the circular hole of the at least one slot causes deformation of the side walls of the cartridge body.

13. The stapling device of claim 9, wherein the distal end portions of the clamp members are secured together with rivets, the rivets including a central portion having a first diameter and opposite end portions having a second diameter smaller than the first diameter.

14. The stapling device of claim 13, wherein the opposite end portions of the rivets form the posts.

15. A stapling device comprising:
a frame assembly defining a longitudinal axis and including spaced frame members, each of the spaced frame members having a proximal portion and a distal portion and defining a frame assembly channel;
a clamp slide assembly supported within the frame assembly channel and including spaced clamp members, each of the spaced clamp members having a distal end portion and at least one post extending outwardly from the respective clamp member; and
a tool assembly including an anvil assembly and a cartridge assembly, the anvil assembly supported on the distal portions of the frame members, the cartridge assembly being releasably supported on the distal end portion of the clamp members and including a cartridge body having spaced side walls, the spaced side walls defining a cartridge channel dimensioned to receive the distal end portions of the spaced clamp members, each of the spaced side walls defining at least one slot that receives the at least one post of the clamp slide assembly when the cartridge assembly is received on the clamp slide assembly to retain the cartridge assembly on the clamp slide assembly, wherein the at least one slot is defined by angled walls that converge in a distal direction into a circular hole, each of the circular holes of the at least one slot includes an entryway having a width, each of the at least one post having a diameter that is larger than the width of the entryway such that receipt of the at least one post within the circular hole of the at least one slot causes deformation of the side walls of the cartridge body.

16. The stapling device of claim 15, wherein the proximal portion of the frame assembly supports a handle assembly.

17. The stapling device of claim 16, wherein the handle assembly includes a stationary handle and a trigger, the trigger movable in relation to the stationary handle to actuate the tool assembly.

18. The stapling device of claim 15, wherein the distal end portions of the clamp members are secured together with rivets, the rivets including a central portion having a first diameter and opposite end portions having a second diameter smaller than the first diameter.

19. The stapling device of claim 18, wherein the opposite end portions of the rivets form the posts.
